(19) United States
(12) Patent Application Publication
(10) Pub. No.: US 2025/0311970 A1
(43) Pub. Date: Oct. 9, 2025

(54) SLEEP ANALYSIS SEGMENT DETECTION METHOD AND SYSTEM

(71) Applicant: BRLAB, INC., Seoul (KR)

(72) Inventors: Hyunbin KWON, Seoul (KR); Jongmin LEE, Seoul (KR); Kwangsuk PARK, Seoul (KR); Byunghun CHOI, Seoul (KR); Jonghyeok JEONG, Seoul (KR)

(21) Appl. No.: 19/245,109

(22) Filed: Jun. 20, 2025

Related U.S. Application Data
(63) Continuation of application No. PCT/KR2023/021431, filed on Dec. 22, 2023.

(30) Foreign Application Priority Data
Dec. 22, 2022 (KR) 10-2022-0182003
Dec. 22, 2022 (KR) 10-2022-0182004
Apr. 10, 2023 (KR) 10-2023-0047110
Dec. 22, 2023 (KR) 10-2023-0189305

(51) Int. Cl. A61B 5/00

(52) U.S. Cl. CPC A61B 5/4815; A61B 5/6892

(57) ABSTRACT

The present disclosure relates to a sleep analysis segment detection method and system, and specifically, a sleep analysis segment detection method according to an embodiment of the present disclosure, which is performed in a system including a layer unit, a detection unit, a memory unit, and a data analysis unit, may include detecting a user's biosignal from the detection unit provided in the layer unit; storing the user's biosignal detected by the detection unit in the memory unit; and analyzing the user's biosignal stored in the memory unit by the data analysis unit to detect the user's sleep analysis segment.

### CROSS-REFERENCE TO RELATED APPLICATIONS

This application is a Continuation of Application No. PCT/KR2023/021431, filed on Dec. 22, 2023, which in turn claims the benefit of Korean Patent Applications No. 10-2022-0182003, filed on Dec. 22, 2022, No. 10-2022-0182004, filed on Dec. 22, 2022, No. 10-2023-0047110, filed on Apr. 10, 2023, and No. 10-2023-0189305, filed on Dec. 22, 2023. The entire disclosures of all these applications are hereby incorporated by reference.

### TECHNICAL FIELD

The present disclosure relates to a sleep analysis segment detection method and system, and more particularly, to a sleep analysis segment detection method and system that collects a user's biosignal positioned on a mattress in a non-contact manner and detects an effective analysis segment required for the user's sleep analysis using the collected user biosignal.

### BACKGROUND ART

Recently, as people's living standards and quality have improved, the demand for ‘good sleep’ has increased, and in particular, the industry that provides various sleep inducing devices or services using the latest scientific technologies is growing significantly. Accordingly, many devices and services are being commercialized to improve sleep quality, such as sleep care services that provide guidance on sleep environment, habits, and posture through consulting with experts, and services that monitor a sleep state by detecting a user's breathing sounds, or the like when wearing a wearable device. In order to provide consulting on ‘good sleep’ to people, polysomnography is often performed to diagnose various normal or abnormal states during the user's sleep.

The polysomnography is a test that collects the user's body information using various devices and instruments during the user's sleep segment, and analyzes the user's complex sleep state based on the collected body information. That is, during a polysomnography test, various examination equipment may be mobilized to diagnose the user's sleep state, and for example, an electroencephalogram (EEG) test to determine a brain function state, an electrooculogram (EOG) test to observe eye movements, an electromyogram (EMG) test to determine muscle condition, an electrocardiogram (ECG) to observe heart rhythm, and a video recording to observe an overall condition are performed together, and the test may usually be conducted while the user sleeps for about one night.

That is, since a polysomnography test is performed by a skilled expert turning the examination equipment on and off at the start and end time points of the user's sleep, a sleep analysis segment called total recording time (TRT) may be determined, and TRT is used to calculate parameters that analyze the user's sleep state. For example, if the user's total sleep time period is divided by the TRT, the user's sleep efficiency may be calculated, and the TRT value may be utilized in calculating various sleep parameters of the user.

However, in the case of polysomnography using the prior art, there is a limitation that an expert who can exclusively operate the examination equipment is absolutely necessary, and even for the analysis segment in which the user's sleep state is to be analyzed, it is necessary to rely on experts to determine the start and end time points of the user's sleep. In other words, in the case of the prior art, it is difficult to define the user's exact TRT value in an ordinary household without an expert, making sleep state analysis impossible, and there is a disadvantage in that the user must visit a specialized institution (hospital, clinic, etc.) where an expert is present to analyze a sleep state.

The present disclosure has been proposed to complement the disadvantage of the prior art, and provides a sleep analysis segment detection method and system that can automatically detect a user's sleep analysis segment and analyze the user's sleep state even in a general home environment without an expert, simply by sleeping in sleep equipment capable of collecting the user's body information.

### DISCLOSURE OF INVENTION

### Technical Problem

An aspect of the present disclosure is to provide a sleep analysis segment detection method and system that can collect a user's biosignal in a non-contact manner and detect an effective analysis segment required for the user's sleep analysis using the collected biosignal.

In addition, an aspect of the present disclosure is to provide a sleep analysis segment detection method and system that can automatically detect a sleep analysis segment required for analyzing a user's sleep state even in a general home environment without the assistance of an expert.

Moreover, an aspect of the present disclosure is to provide a sleep analysis segment detection method and system that can automatically distinguish a user's sleep activity and non-sleep activity and detect a valid sleep analysis segment required for analyzing a sleep state.

Meanwhile, technical problems of the present disclosure are not limited to the above-mentioned problems, and other technical problems which are not mentioned herein will be clearly understood by those skilled in the art from the description below.

### Technical Solution

A sleep analysis segment detection method according to the present disclosure, which is a sleep analysis segment detection method performed in a system including a layer unit, a detection unit, a memory unit, and a data analysis unit, may include detecting a user's biosignal from the detection unit provided in the layer unit; storing the user's biosignal detected by the detection unit in the memory unit; and analyzing the user's biosignal stored in the memory unit by the data analysis unit to detect the user's sleep analysis segment.

In addition, in the sleep analysis segment detection method according to the present disclosure, the detecting of the user's biosignal may include performing detection on at least one information of user's weight, height, body proportions, identification information, movement information, heart rate, and breathing state through the detection unit including at least one sensor among a pressure sensor, a vibration sensor, a piezoelectric sensor, an acceleration sensor, an acoustic sensor, a polyvinylidene film (PVDF) sensor, an electromechanical film (EMFi) sensor, a force sensing resistor (FSR) sensor, an infrared sensor, a motion sensor, and a facial recognition sensor.

In addition, in the sleep analysis segment detection method according to the present disclosure, the storing of the user's biosignal in the memory unit may be performed so as to allow the user's biosignal detected by the detection unit to be mapped and stored for each user.

In addition, in the sleep analysis segment detection method according to the present disclosure, the detecting of the user's sleep analysis segment from the data analysis unit may include a first sleep analysis segment detection step of detecting a time point when the user lies down on the layer unit and a time point when the user gets up and leaves the layer unit.

In addition, in the sleep analysis segment detection method according to the present disclosure, the first sleep analysis segment detection step may be performed by utilizing the user's biosignal detected by at least one sensor among a pressure sensor, a vibration sensor, a piezoelectric sensor, an acceleration sensor, a polyvinylidene film (PVDF) sensor, an electromechanical film (EMFi) sensor, and a force sensing resistor (FSR) sensor in the detection unit.

In addition, in the sleep analysis segment detection method according to the present disclosure, the method may further include, subsequent to the first sleep analysis segment detection step, a second sleep analysis segment detection step of excluding the user's non-sleep segment from the first sleep analysis segment.

In addition, in the sleep analysis segment detection method according to the present disclosure, the second sleep analysis segment detection step may include determining a segment in which at least one of the user's heart rate, breathing state, and movement information detected by the detection unit exceeds a preset threshold value as a non-sleep segment, and excluding the non-sleep segment from the first sleep analysis segment.

In addition, in the sleep analysis segment detection method according to the present disclosure, the threshold value may refer to a biosignal value that is determined to be in a non-sleep state while the user is lying on the layer unit, and may be set for each user.

In addition, in the sleep analysis segment detection method according to the present disclosure, the method may further include, subsequent to the second sleep analysis segment detection step, a third sleep analysis segment detection step of excluding the user's activity segment from the second sleep analysis segment.

In addition, in the sleep analysis segment detection method according to the present disclosure, the third sleep analysis segment detection step may include determining a segment in which a signal value detected from the pressure sensor of the detection unit remains lower than a preset threshold value for above a preset time period as a user activity segment, and excluding the user activity segment from the second sleep analysis segment.

In addition, in the sleep analysis segment detection method according to the present disclosure, the threshold value may refer to a biosignal value determined to be a state in which the user has gotten up and left the layer unit, and may be set for each user.

In addition, in the sleep analysis segment detection method according to the present disclosure, the method may further include, subsequent to the third sleep analysis segment detection step, summing up, when there are at least two or more multiple independent sleep analysis segments within the third sleep analysis segment, the multiple sleep analysis segments.

In addition, in the sleep analysis segment detection method according to the present disclosure, the detecting of the user's biosignal from the detection unit may be performed from a preset analysis start time point to a preset analysis end time point, or from a predetermined time period prior to the preset analysis start time point to a predetermined time period subsequent to the preset analysis end time point.

In addition, in the sleep analysis segment detection method according to the present disclosure, a predetermined time period prior to the preset analysis start time point and a predetermined time period subsequent to the preset analysis end time point may be automatically adjusted based on the accumulated user's biosignal or the user's biosignal being detected.

In addition, in the sleep analysis segment detection method according to the present disclosure, the method may further include storing the detected user's sleep analysis segment in the memory unit subsequent to detecting the user's sleep analysis segment.

In addition, in the sleep analysis segment detection method according to the present disclosure, the storing of the detected users' sleep analysis segment in the memory unit may be performed to allow the sleep analysis segment to be mapped and stored for each user.

A sleep analysis segment detection system according to the present disclosure may include at least one layer having a plurality of components; a detection unit provided in the layer unit to detect a user's biosignal; a memory unit in which the user's biosignal detected by the detection unit is stored; and a data analysis unit that analyzes the user's biosignal stored in the memory unit to detect the user's sleep analysis segment.

In addition, in the sleep analysis segment detection system according to the present disclosure, the detection unit may include at least one sensor among a pressure sensor, a vibration sensor, a piezoelectric sensor, an acceleration sensor, an acoustic sensor, a polyvinylidene film (PVDF) sensor, an electromechanical film (EMFi) sensor, a force sensing resistor (FSR) sensor, an infrared sensor, a motion sensor, and a facial recognition sensor.

In addition, in the sleep analysis segment detection system according to the present disclosure, at least one information of the user's weight, height, body proportions, identification information, movement information, a heart rate, a breathing state, a sleep set time period, a sleep analysis segment, and a threshold value may mapped and stored for each user in the memory unit.

In addition, in the sleep analysis segment detection system according to the present disclosure, the user's sleep analysis segment detected by the data analysis unit may be a second sleep analysis segment detected by excluding the user's non-sleep segment from a first sleep analysis segment detected by sensing a time point when the user lies down on the layer unit and a time point when the user gets up and leaves the layer unit, and a third sleep analysis segment detected by excluding the user's active segment from the second sleep analysis segment.

In addition, in the sleep analysis segment detection system according to the present disclosure, the user's sleep analysis segment detected by the data analysis unit may be a sleep analysis segment in which at least two or more respectively independent sleep analysis segments that are present within the third sleep analysis segment are summed up together.

### Advantageous Effects

According to the present disclosure, a user's biosignal may be collected in a non-contact manner, and an effective analysis segment required for the user's sleep analysis may be automatically detected using the collected biosignal.

In addition, according to the present disclosure, a sleep analysis segment required for analyzing a user's sleep state may be automatically detected even in a general home environment, thereby allowing the user to easily detect a sleep analysis segment without the assistance of an expert or specialized institution.

Moreover, according to the present disclosure, even when the user's sleep time period is not manually monitored, the user's sleep activity and non-sleep activity may be automatically distinguished to more accurately detect a valid sleep analysis segment required for analyzing a sleep state.

Meanwhile, the effects of the present disclosure may not be limited to the above-mentioned effects, and other technical effects which are not mentioned herein will be clearly understood by those skilled in the art from the description below.

### BRIEF DESCRIPTION OF THE DRAWINGS

FIG. 1 is a diagram for explaining a sleep analysis segment detection system according to an embodiment of the present disclosure.

FIG. 2 is a diagram for explaining a sleep analysis segment detection method according to an embodiment of the present disclosure.

FIG. 3 is a diagram for more specifically explaining a sleep analysis segment detection process according to an embodiment of the present disclosure.

FIG. 4 is a diagram for explaining a first sleep analysis segment detection step according to an embodiment of the present disclosure.

FIG. 5 is a diagram for explaining a second sleep analysis segment detection step according to an embodiment of the present disclosure.

FIG. 6 is a diagram for explaining a third sleep analysis segment detection step according to an embodiment of the present disclosure.

FIG. 7 is a diagram for explaining a sleep analysis segment detection method according to another embodiment of the present disclosure.

FIG. 8 is a diagram for explaining a memory unit of a sleep analysis segment detection system according to still another embodiment of the present disclosure.

FIG. 9 is a diagram for explaining a sleep analysis segment detection method according to still another embodiment of the present disclosure.

### BEST MODE FOR CARRYING OUT THE INVENTION

The details of the objects and technical configurations of the present disclosure and operational effects thereof will be more clearly understood from the following detailed description based on the accompanying drawings appended hereto. Hereinafter, embodiments according to the present disclosure will be described in detail with reference to the accompanying drawings.

Embodiments disclosed herein should not be interpreted as limiting or used to limit the scope of the present disclosure. It is apparent for those skilled in the art that a description including embodiments herein has various applications. Therefore, any embodiments described in the detailed description of the present disclosure are illustrative for better understanding of the present disclosure and are not intended to limit the scope of the present disclosure to the embodiments.

Functional blocks illustrated in the drawings and described hereunder are only examples of possible implementations. In other implementations, other functional blocks may be used without departing from the concept and scope of the detailed description. Furthermore, one or more functional blocks of the present disclosure are illustrated as separate blocks, but one or more of the functional blocks of the present disclosure may be a combination of various hardware and software elements that execute the same function.

In addition, an expression that some elements are “included” is an expression of an “open type,” and the expression simply denotes that the elements are present, but should not be construed as excluding additional elements. Moreover, in case where it is mentioned that one element is “connected” or “coupled” to the other element, it should be understood that one element may be directly connected to the other element, but another element may be present therebetween.

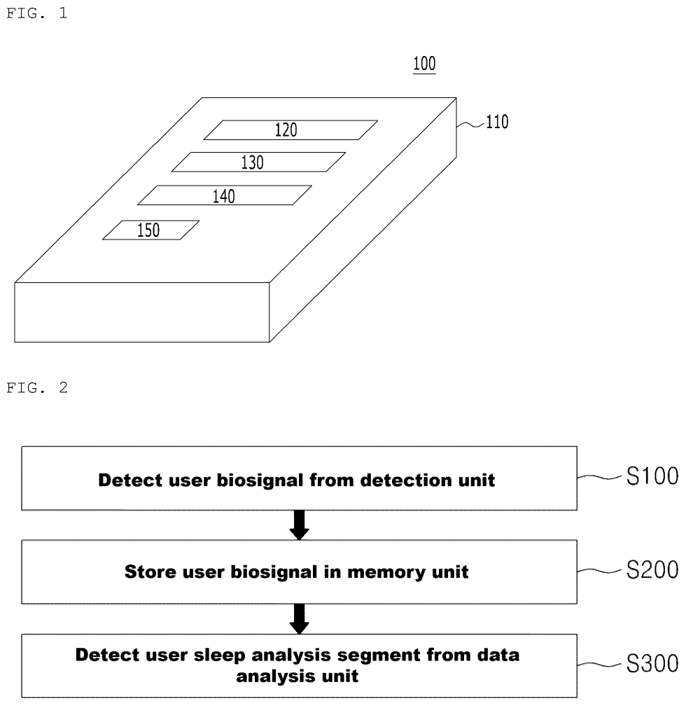

FIG. 1 is a diagram for explaining a sleep analysis segment detection system according to an embodiment of the present disclosure.

Referring to FIG. 1, a sleep analysis segment detection system 100 according to the present disclosure includes at least one layer unit 110 having a plurality of components, a detection unit 120 provided in the layer unit 110 to detect a user's biosignal, a memory unit 130 in which the user's biosignal detected by the detection unit 120 is stored, and a data analysis unit 140 that analyzes the user's biosignal stored in the memory unit 130 to detect the user's sleep analysis segment. In addition, the sleep analysis segment detection system 100 according to the present disclosure may further include a component of a communication unit 150 that is connected to the system 100 and an external device or server in a wired/wireless manner to transmit and receive information such as data, a power supply unit (not shown) that controls the on/off and power of the system 100, and a control unit (not shown) that controls the components of the system 100.

The layer unit 110 may be understood as a member having a receiving space in which the components to be described later can be arranged, and if a receiving space is provided, there is no limitation on the material or shape of the layer unit 110. The layer unit 110 may be a space where a user sleeps, for example, a mattress, or may be any one of a plurality of surfaces constituting a mattress. Additionally, the layer unit 110 may be a mat that may be placed on a mattress, and may furthermore be a member made of wood or metal rather than cotton with a fiber material. In this manner, if the layer unit 110 has a predetermined receiving space, there is no limitation on the material or shape. However, in order to help understand the disclosure, in this detailed description, the explanation will be continued assuming that the layer unit 110 is a mattress.

The detection unit 120, which is provided on the layer unit 110, is configured to detect and acquire a biosignal from a user. The detection unit 120 may include one of various types of sensors capable of detecting a user's biosignal, for example, a pressure sensor, a vibration sensor, a piezoelectric sensor, an acceleration sensor, an acoustic sensor, a polyvinylidene film (PVDF) sensor, an electromechanical film (EMFi) sensor, a force sensing resistor (FSR) sensor, an infrared sensor, a motion sensor, and a facial recognition sensor, and may be configured with a combination of at least one or more of the sensors.

Specifically, the detection unit 120 may detect the user's operation of lying on or getting up from the layer unit 110 or the user's weight or location information through a pressure sensor, or may detect the user's vibration signal through a vibration sensor to acquire state information such as a heart rate, a breathing state, and a movement state. In addition, the detection unit 120 may recognize the user's voice through an acoustic sensor, recognize the user's face through a facial recognition sensor, and detect the user's gesture through a motion sensor to receive feedback information.

In this way, the detection unit 120 of the present disclosure may be a component for detecting various biosignals of a user located on the layer unit 110, and adjusting the type, number, layout, and the like of sensors used to detect more precise and specific user state information cannot be limited to an embodiment of the present disclosure.

The memory unit 130, which is a component in which the user's biosignal detected by the detection unit 120 is stored, may be provided by being built into the sleep analysis segment detection system 100 or may be an external device connected to the sleep analysis segment detection system 100 through a wired or wireless communication manner. The type or location of the memory unit 130 cannot be limited to an embodiment of the present disclosure.

In the memory unit 130, detected user's biosignal may be mapped and stored for each user. For example, in addition to body information such as weight, height, and body proportions of user A, information such as facial information and voice information may be mapped and stored in the memory unit 130 with identification information that can identify the user, and furthermore, more specific information such as heart rate information, movement information, a breathing state, a sleep analysis segment, and a threshold values required for analyzing sleep analysis segment of user A may be mapped and stored. In addition, a sleep set time period that is preset for each user may be mapped and stored in the memory unit 130, and the sleep set time period may be automatically or manually adjusted and stored based on the user information stored in the memory unit 130.

The data analysis unit 140 is configured to detect the user's sleep analysis segment based on the user's biosignal stored in the memory unit 130. For reference, the data analysis unit 140 may also be understood as a central processing unit. The central processing unit may also be referred to as a controller, a microcontroller, a microprocessor, a microcomputer, or the like. Furthermore, the central processing unit may be implemented by hardware or firmware, software, or a combination thereof, and configured to include an application specific integrated circuit (ASIC) or a digital signal processor (DSP), a digital signal processing device (DSPD), a programmable logic device (PLD), or a field programmable gate array (FPGA) when implemented using hardware, and configured with firmware or software to include a module, a procedure, a function or the like that performs the foregoing functions or operations when implemented using firmware or software.

Here, the user's sleep analysis segment detected by the data analysis unit 140 according to an embodiment of the present disclosure may include a first sleep analysis segment detected by sensing a time point when the user lies down on the layer unit 110 and a time point when the user gets up and leaves the layer unit 110, a second sleep analysis segment detected by excluding the user's non-sleep segment from the first sleep analysis segment, and a third sleep analysis segment detected by excluding the user's activity segment from the second sleep analysis segment, and preferably, the last third sleep analysis segment may be a sleep analysis segment finally detected by the data analysis unit 140. In addition, the user's sleep analysis segment detected by the data analysis unit 140 may be a sleep analysis segment in which at least two or more multiple independent sleep analysis segments that are present within the third sleep analysis segment are summed up.

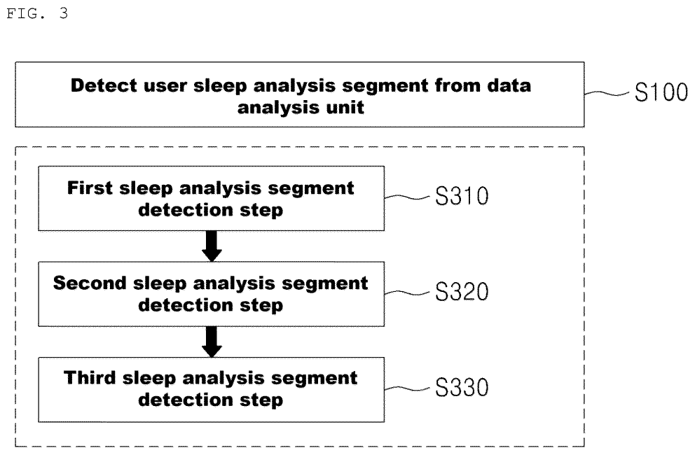

Hereinafter, a method of detecting a user's sleep analysis segment according to an embodiment of the present disclosure will be described with reference to FIGS. 2 to 9.

FIG. 2 is a diagram for explaining a sleep analysis segment detection method according to the present disclosure, and referring to FIG. 2, a sleep analysis segment detection method performed in a system including a layer unit, a detection unit, a memory unit, and a data analysis unit, may include detecting a user's biosignal from the detection unit provided in the layer unit (S100), storing the user's biosignal detected by the detection unit in the memory unit (S200), and analyzing the user's biosignal stored in the memory unit by the data analysis unit to detect the user's sleep analysis segment (S300).

The detecting of the user's biosignal (S100) is performed to detect at least one information of a user's weight, height, body proportions, identification information, movement information, heart rate, and breathing state through the detection unit including at least one sensor among a pressure sensor, a vibration sensor, a piezoelectric sensor, an acceleration sensor, an acoustic sensor, a polyvinylidene film (PVDF) sensor, an electromechanical film (EMFi) sensor, a force sensing resistor (FSR) sensor, an infrared sensor, a motion sensor, and a facial recognition sensor, and the storing of the user's biosignal in the memory unit (S200) is performed to allow the user's biosignal detected by the detection unit to be mapped and stored for each user.

That is, according to an embodiment of the present disclosure, since the user's biosignals measured from the sensors of the detection unit are mapped for each user and stored in the memory unit, when the detection unit detects the user's biosignal, the user may be identified based on the detected biosignal, and even the user's state information (e.g., whether the user is in a sleep state, a non-sleeping state, an activity state, etc.) may be determined, and accordingly, the present disclosure may automatically detect the user's valid sleep analysis segment.

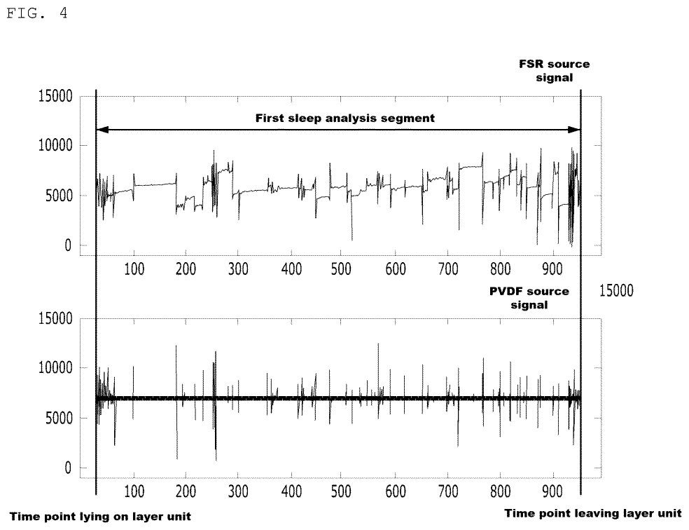

FIG. 3 is a diagram for more specifically explaining a sleep analysis segment detection process according to an embodiment of the present disclosure, and referring to FIG. 3, the detecting of a user sleep analysis segment in the data analysis unit (S300) may be performed by including at least one of a first sleep analysis segment detection step (S310), a second sleep analysis segment detection step (S320), and a third sleep analysis segment detection step (S330).

The first sleep analysis detection step (S310) is performed in a manner of sensing a time point when the user lies down on the layer unit and a time point when the user gets up and leaves the layer unit, and detecting a first sleep analysis segment using them as a start time point and an end time point, respectively. The time point when the user lies down on the layer unit and the time point when the user gets up and leaves the layer unit may be performed by utilizing the user's biosignal detected by at least one sensor among a pressure sensor, a vibration sensor, a piezoelectric sensor, an acceleration sensor, a polyvinylidene film (PVDF) sensor, an electromechanical film (EMFi) sensor, and a force sensing resistor (FSR) sensor in the detection unit.

That is, the present disclosure may not detect a user's sleep analysis segment solely based on a start time point and an end time point set by a user or automatically/manually by a system, but rather detect the user's first sleep analysis segment by using a time point when the user actually lies down or gets up on the layer unit as the start time point and end time point of the sleep analysis segment based on the user's biosignal detected by the detection unit, thereby allowing a more accurate sleep analysis segment to be detected without a separate user operation.

Next, a second sleep analysis segment detection step (S320) may be performed subsequent to the first sleep analysis segment detection step (S310), and the second sleep analysis segment detection step (S320) may be performed in a manner of excluding the user's non-sleep segment from the first sleep analysis segment. Here, the user's non-sleep segment refers to a segment in which the user engages in activities unrelated to sleep analysis even after lying on the layer unit, such as looking at a smartphone, reading, or making a phone call while lying in bed without falling asleep, and the non-sleep segment may be determined based on the user's biosignal information, such as the user's heart rate, breathing state, movement information, and voice information, detected by the detection unit. In the present disclosure, the non-sleep segment may be automatically excluded from the first sleep analysis segment, thereby detecting a more accurate second sleep analysis segment.

Next, subsequent to the second sleep analysis segment detection step (S320), a third sleep analysis segment detection step (S330) that excludes the user's activity segment from the second sleep analysis segment may be performed. Here, the user's activity segment refers to a segment in which the user gets up before or during sleep to go to the bathroom or briefly leaves sleep activity, and the activity segment may be determined based on a pressure signal or vibration signal detected from the pressure sensor of the detection unit. In the present disclosure, the user activity segment may be automatically excluded from the second sleep analysis segment, thereby detecting a more accurate third sleep analysis segment.

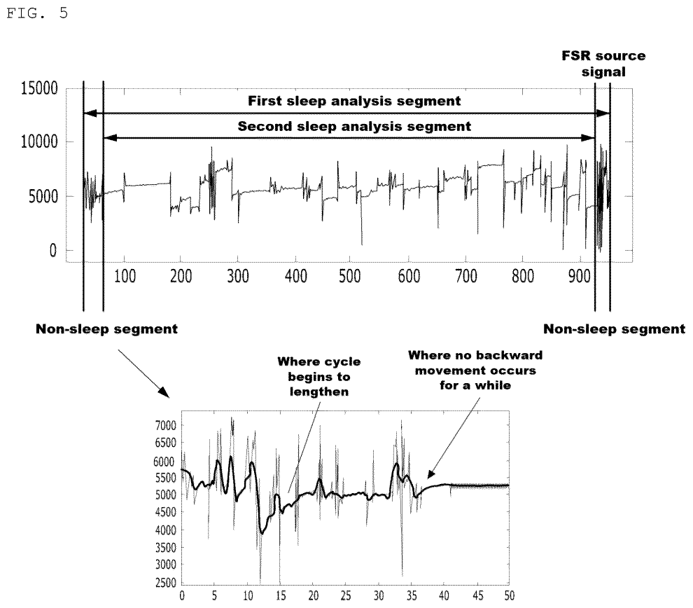

Hereinafter, the first to third sleep analysis segment detection steps according to an embodiment of the present disclosure will be described in more detail with reference to FIGS. 4 to 6.

FIG. 4 is a diagram for explaining a first sleep analysis segment detection step according to an embodiment of the present disclosure.

Referring to FIG. 4, the first sleep analysis segment detection step is performed by utilizing a user's biosignal detected by at least one sensor among a pressure sensor, a vibration sensor, a piezoelectric sensor, an acceleration sensor, a polyvinylidene film (PVDF) sensor, an electromechanical film (EMFi) sensor, and a force sensing resistor (FSR) sensor of the detection unit, and a time point when the user lies down on the layer unit is set as a start time point, and a time point when the user gets up and leaves the layer unit is set as an end time point.

As shown in FIG. 4, the sleep analysis segment detection system of the present disclosure determines when a signal value of a pressure sensor or a piezoelectric sensor, for example, a force sensing resistor (FSR) sensor or a PVDF sensor, detected by a detection unit of a layer unit exceeds a threshold value, for example, “0,” so as to be detected as a numerical value corresponding to the user's body weight as a time point when the user lies down on the layer unit, and defines it as a “start time point,” and determines when the signal value becomes the threshold value, for example, “0,” or falls below “0” so as to be detected as a numerical value indicating that the user is not present as a time point when the user gets up and leaves the layer unit, and defines it as an “end time point,” thereby detecting the segment from the “start time point” to the “end time point” as a first sleep analysis segment.

That is, according to the present disclosure, there is no need for the user to directly set the start and end of the sleep analysis detection segment or for an expert to operate the equipment, and it is determined, based on the user's biosignal detected by the detection unit, whether the user lies down on the layer unit or the user gets up from the layer unit, and the start and end time points of the first sleep analysis segment may be automatically set.

FIG. 5 is a diagram for explaining a second sleep analysis segment detection step according to an embodiment of the present disclosure.

The second sleep analysis segment detection step may be performed in a manner of excluding the user's “non-sleep segment” from the first sleep analysis segment of FIG. 4. Here, the user's “non-sleep segment” refers to a segment in which the user engages in activities unrelated to sleep analysis even after lying on the layer unit, such as looking at a smartphone, reading, or making a phone call while lying in bed without falling asleep, and the “non-sleep segment” may be determined based on the user's biosignal information, such as the user's heart rate, breathing state, movement information, and voice information, detected by the detection unit.

Specifically, a segment in which at least one biosignal among the user's heart rate, breathing state, and movement information detected by the detection unit exceeds a preset threshold value may be determined as a “non-sleep segment,” and the threshold value refers to a biosignal value determined to be in a “non-sleep state” while the user is lying on the layer unit, and may be set for each user.

That is, as shown in FIG. 5, based on indicators of where the cycle of the biosignal values or the cycle of the user's movement occurrence (which can be inferred from the biosignal values) during the user's first sleep analysis segment begins to lengthen, when the user's breathing is fast or irregular, or when the heartbeat is fast or irregular, or when the movement occurs frequently or periodically, it may be determined as a “non-sleep segment,” and when detected to be above a predetermined threshold value for each user, which is set based on the user's biosignal values stored in the memory unit, it may be determined as a “non-sleep segment” in which the user engages in other activities before entering a sleep state.

For example, based on the biosignal values of user A stored in the memory unit, a sleep state threshold value of user A may be set to a range of 5,000 to 5,500, and a segment in which a user movement index (or an index value calculated through at least two or more indicators) detected by the detection unit becomes longer than the threshold value may be determined as a start time point of the user's “non-sleep segment,” and a point at which the user's movement index value is determined to be within the threshold range and the user's movement does not occur for a while may be determined as an end time point of the “non-sleep segment,” thereby detecting a second sleep analysis segment by excluding the “non-sleep segment” from the user's first sleep analysis segment.

The threshold value used to define the “non-sleep segment” refers to a biosignal value that is determined to be in a non-sleep state while the user is lying on the layer but is not in a sleep state, and may be set differently for each user. Additionally, the threshold value may be set based on the user's past sleep or non-sleep state biosignal values, and may be automatically adjusted by accumulating and storing the user's biosignal and updating periodically/aperiodically, or may be manually readjusted by the user.

Therefore, the present disclosure may not simply detect an entire time period in which a user is lying in bed as a sleep analysis segment, but automatically exclude a “non-sleep segment” in which the user is lying in bed but is determined to be in a non-sleep state, such as looking at a smartphone or reading, from the sleep analysis segment, thereby detecting a second sleep analysis segment that can be more effectively utilized in analyzing the user's actual sleep state.

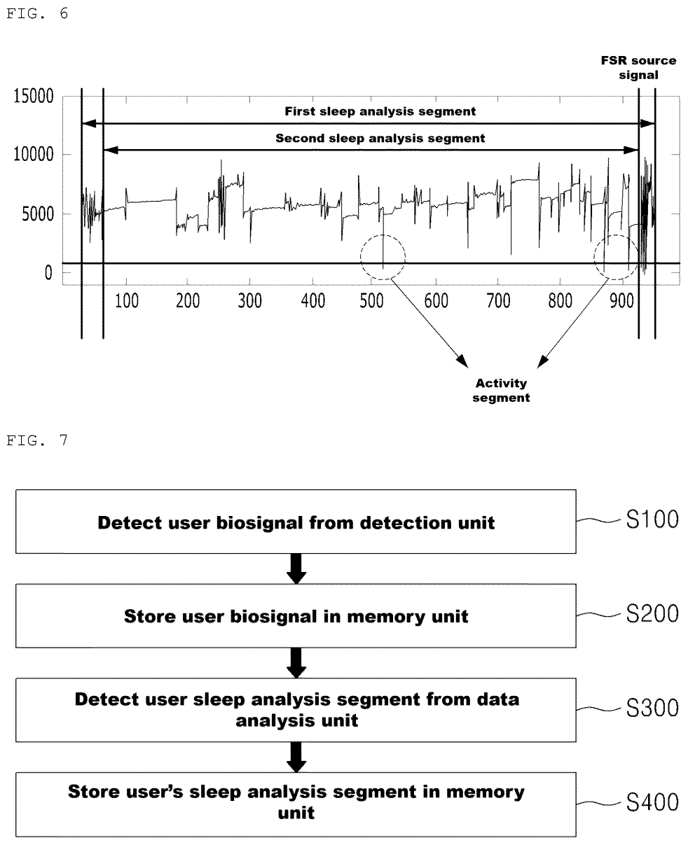

FIG. 6 is a diagram for explaining a third sleep analysis segment detection step according to an embodiment of the present disclosure.

Referring to FIG. 6, the third sleep analysis segment detection step may be performed in a manner of excluding the user's “activity segment” from the second sleep analysis segment of FIG. 5. Additionally, although not shown, the third sleep analysis segment detection step may also be performed in a manner of excluding the user's “activity segment” from the first sleep analysis segment of FIG. 4.

Here, the “activity segment” of the user refers to a segment in which the user gets up before or during sleep and briefly leaves sleep activity, such as going to the bathroom or watching TV, and whether or not to exclude the “activity segment” from the sleep analysis segment may be determined based on a pressure signal or vibration signal detected from the pressure sensor of the detection unit.

Specifically, a portion in which a signal value detected from the pressure sensor of the detection unit falls below a predetermined threshold value is determined as a state where the user has left the layer unit, and a segment in which a state of falling below a preset threshold value is maintained for above a preset time period may be determined as a user “activity segment.”

If the signal value detected from the pressure sensor of the detection unit is below the threshold value, for example, “0” or falls below “0,” the user is determined to get up from the layer unit, and when this state is maintained within a predetermined threshold time period (e.g., 30 to 60 seconds), it may be defined as an activity that does not significantly disturb the user's sleep state, such as briefly going to the bathroom, and thus may be included in the sleep analysis segment, and when this state is maintained for above a predetermined threshold time period (for example, above 60 seconds), it may be defined as an “activity segment” unrelated to the sleep state, such as leaving the sleep state and staying in the bathroom for a long time or watching TV in the living room, and thus excluded from the sleep analysis segment.

The threshold value used to define the “activity segment” may refer to a biosignal value at which the user is determined to get up from the layer and leave the sleep state, and may be set differently for each user. Additionally, the threshold value may be set based on the user's past sleep or non-sleep state biosignal values, and may be automatically adjusted by accumulating and storing the user's biosignal and updating periodically/aperiodically, or may be manually readjusted by the user.

Accordingly, the present disclosure may detect a third sleep analysis segment that can be more effectively utilized for analyzing the user's actual sleep state by automatically excluding an “activity segment” in which the user gets out of bed during a sleep state and is disturbed by the sleep state or is unrelated to the sleep state, from the sleep analysis segment.

In addition, although not shown, subsequent to the third sleep analysis segment detection step, when there are at least two or more multiple independent sleep analysis segments within the detected third sleep analysis segment, the multiple sleep analysis segments may be summed up to detect a final sleep analysis segment.

That is, in a process of detecting the user's sleep analysis segment, each independent sleep segment may be distinguished and included before or after segments defined as included in or excluded from the sleep analysis segment, and when there are multiple independent sleep segments within an entire sleep analysis segment, a final sleep analysis segment may be detected by summing up each independent sleep segment, or the sleep analysis segment detection process may be performed again by retrieving all data from the pressure sensor and the vibration sensor at the start time point of the first independent sleep to the pressure sensor and vibration sensor data at the end time point of the last independent sleep.

In this way, a sleep state may be analyzed by looking at data from an entire sleep analysis segment and analyzing the sleep state that reflects the continuity of sleep. For example, a proportion of deep sleep among an entire sleep analysis segment may be concentrated mainly from the start time point of the sleep analysis segment to the first third, and when looking at and analyzing data for the entire sleep analysis segment, the accuracy of the data may be improved by detecting the final sleep analysis segment by summing up each independent sleep segment.

As described above, in detecting a sleep analysis segment required for analyzing a user's sleep state, the present disclosure has the following features: 1) a time point when lying on or getting up from bed is automatically detected without any separate operation by the user; 2) a non-sleep segment in which the user engages in activities unrelated to the sleep state even while lying in bed is automatically excluded; and 3) a segment in which the user leaves the bed for a predetermined time period and engages in activities that interfere with the sleep state is automatically excluded, and the sleep analysis segment detection process may be automatically performed by detecting the user's biosignal without manual operation by the user.

Accordingly, the present disclosure may automatically detect start and end time points of a user's sleep and automatically detect a sleep analysis segment simply by the user lying on a bed on which a sleep analysis segment detection method according to the present disclosure is executed, and automatically exclude a non-sleep segment on the bed before sleeping and an activity segment leaving the bed during sleep from the sleep analysis segment, and therefore, it has an advantage of automatically detecting a valid sleep analysis segment required for analyzing the user's sleep state even in a general home environment where there is no expert or specialized equipment.

In particular, the present disclosure may automatically determine the user's non-sleep segment and activity segment by utilizing a threshold value set based on the user's biosignal value in a sleep and non-sleep state, as well as identify the user simply by the user's action of lying on the bed, and may obtain an effect of automatically detecting the user's sleep analysis segment more accurately and easily even without separate operation or setting by the user.

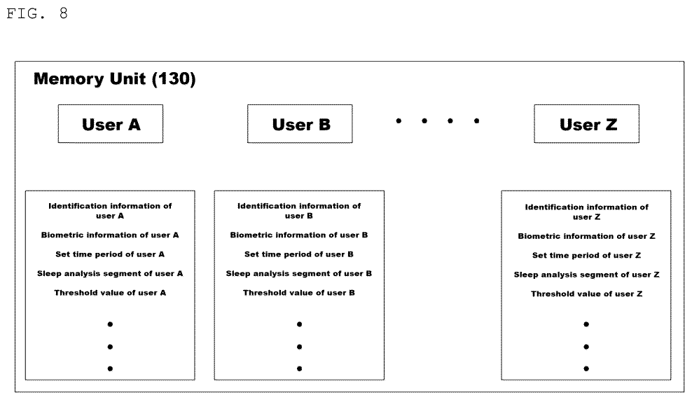

To this end, a process is required in which the user's biosignal and detected sleep analysis segments are updated and accumulated in the memory, and hereinafter, a sleep analysis segment detection method according to another embodiment of the present disclosure will be described with reference to FIG. 7.

As shown in FIG. 7, a sleep analysis segment detection method according to another embodiment of the present disclosure includes detecting a user's biosignal from a detection unit provided in a layer unit (S100), storing the user's biosignal detected by the detection unit in a memory unit (S200), analyzing the user's biosignal stored in the memory unit by the data analysis unit to detect the user's sleep analysis segment (S300), and storing the detected user's sleep analysis segment in the memory unit (S400).

That is, according to the present disclosure, the user's biosignal detected by the detection unit may not only be mapped for each user and stored in the memory unit, but also a biosignal threshold value for each user's state (sleep state, non-sleep state, activity state, etc.) defined based on the user's biosignal and the detected user's sleep analysis segment may be mapped to the user and stored again in the memory unit, and accordingly, the information stored in the memory unit may be utilized when determining the user's sleep/non-sleep state and activity state or detecting the user's valid sleep analysis segment. Accordingly, the present disclosure may detect a user's sleep analysis segment based on user information accumulated and stored in the memory unit, thereby having an effect of allowing more accurate detection of a sleep analysis segment suitable for the user's condition.

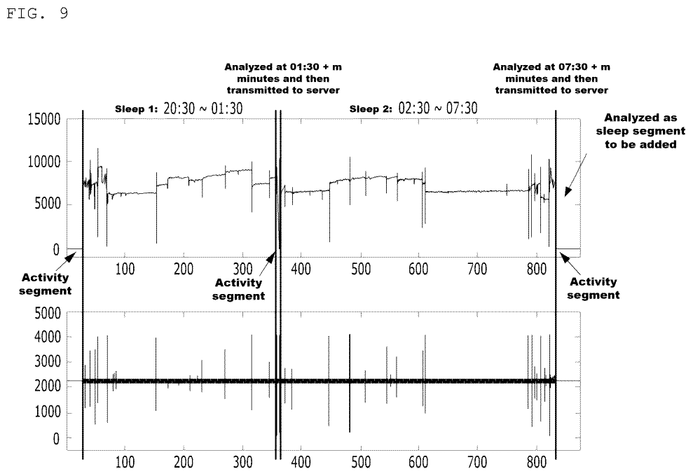

FIG. 8 is a diagram for explaining a memory unit of a sleep analysis segment detection system according to still another embodiment of the present disclosure, and as shown in FIG. 8, at least one or more user information items may be stored in the memory unit 130 of the sleep analysis segment detection system according to the present disclosure.

In the memory unit 130, information of multiple users

may be mapped and stored for each user, and for example, information mapped and stored for user A may include identification information that can distinguish user A, a biosignal of user A, a set time period of user A, a sleep analysis segment of user A, a threshold value of user A, and the like, and those information values may be mapped and stored for other users B, C, and multiple users. Here, the threshold value of user A refers to a threshold value that is used as an indicator to determine the user's sleep state, non-sleep state, activity state, and the like based on the user's biosignal value, and the set time period of user A refers to a set time period that the user can designate as the start and end of the sleep analysis segment.

Therefore, according to the present disclosure, based on an information value mapped and stored for each user in the memory unit 130, the user's sleep state, non-sleep state, activity state, and the like may be more accurately determined, thereby automatically and more accurately detecting an actual valid sleep analysis segment required for sleep analysis among the user's sleep segment.

Furthermore, as another embodiment of the present disclosure, even when there is a sleep analysis set time period specified by the user or automatically set by the system, the user's biosignal may be detected and adjusted to the set time period.

FIG. 9 is a diagram for explaining a sleep analysis segment detection method according to still another embodiment of the present disclosure, and hereinafter, a set time period adjustment process according to another embodiment of the present disclosure will be described in more detail with reference to FIG. 9.

The “set time period” refers to a segment that measures data when detecting the user's sleep analysis segment, and may be a time period directly set by the user in advance to use for detecting his or her own sleep analysis segment, or a time period automatically set by the system based on the user's information stored in the memory unit. That is, basically, the “set time period” is utilized when detecting the user's sleep analysis segment, but in another embodiment of the present disclosure, even if a designated “set time period” is present, the “set time period” may be reasonably adjusted according to the user's biosignal detected by the detection unit.

In other words, when there is a segment determined to be an activity segment before or after or in the middle of the “set time period,” the activity segment may be excluded when detecting the user's sleep analysis segment even if it is included in the “set time period,” and when there is a segment determined to be a sleep segment before or after or in the middle of the “set time period,” the sleep segment may be included and detected in the user's sleep analysis segment even if it is not included in the “set time period.” That is, even if a given user's “set time period” is pre-designated, the user's sleep analysis segment detection process may be performed from several minutes to several tens of minutes before the start of a first independent sleep or from several minutes to several tens of minutes after the end of a last independent sleep.

For example, assuming that a given user's “set time period” is set to 21:00 to 07:00 the next day, the user's sleep state, non-sleep state, and activity state may be determined by detecting the user's biosignal before or after the “set time period,” and thus a sleep segment may be present from 20:30 to 01:30 the next day, and a sleep segment may also present from 02:30 to 07:30. In this case, according to a sleep analysis segment detection method according to another embodiment of the present disclosure, sleep may be analyzed at 1:30+m minutes to be transmitted for a first time, and sleep may be analyzed at 07:30+m minutes to be transmitted for a second time. Meanwhile, according to another embodiment of the present disclosure, data values transmitted for the first and second times may be summed up to or overwritten with the user's biosignal value detected at a pre-specified “set time period” and stored and may be utilized when detecting the user's sleep analysis segment.

In this case, segments between 20:30 and 21:00 and 07:30 and 08:00 may be included and analyzed in the sleep analysis segment of the “set time period” (21:00 to 07:00 the next day) even though they are earlier than the start time point (21:00) and later than the end time point (07:00) of the specified “set time period,” and it can be seen that an entire segment in which the user's sleep can actually be analyzed is 20:30 to 07:30 the next day.

Accordingly, according to another embodiment of the present disclosure, even when a “set time period” for detecting a sleep analysis segment is specified by a user or a system, the user's biosignal detection may be performed from a start time point of the preset “set time period” to a preset analysis end time point, or from a time point that is a predetermined time period prior to the start time point of the preset “set time period” to a time point that is a predetermined time period subsequent to the end time point of the preset “set time period.” In addition, according to another embodiment of the present disclosure, based on the accumulated user's biosignal or the user's biosignal being detected, the “set time period” may be automatically adjusted to a time point that is a predetermined time period prior to the start time point of the preset “set time period” and a time point that is a predetermined time period subsequent to the end time point of the preset “set time period.”

Meanwhile, although not shown, a biosignal may be continuously detected while the user is lying on the layer, and through this, when there is a sleep segment outside of the designated “set time period,” it may be stored in the memory unit as a separate independent sleep. For example, when a user takes a nap and an independent sleep segment is detected between 16:00 and 17:00, which is determined to be a sleep state, it may be stored as a separate sleep segment not included in the designated “set time period” or a sleep segment added to the sleep analysis segment detected at the “set time period” and used as a biosignal indicator required for detecting the user's sleep analysis segment.

In the above, a sleep analysis segment detection method and system according to the present disclosure has been described. Meanwhile, the present disclosure is not limited to the foregoing specific embodiments and application examples, it will be of course understood by those skilled in the art that various modifications may be made without departing from the gist of the present disclosure as defined in the following claims, and it is to be noted that those modifications should not be understood individually from the technical concept and prospect of the present disclosure.

In particular, configurations that implement the technical features of the present disclosure included in the block diagrams and flowcharts shown in the drawings attached to this specification represent logical boundaries between the configurations. However, according to an embodiment of software or hardware, the shown configurations and functions thereof are executed in the form of stand-alone software modules, monolithic software structures, codes, services, and combinations thereof, and the functions may be implemented by being stored in a medium executable on a computer provided with a processor capable of executing the stored program codes, instructions, and the like, and therefore, all of these embodiments should also be regarded as falling within the scope of the present disclosure.

Accordingly, the accompanying drawings and technologies thereof describe the technical characteristics of the present disclosure, but should not be simply inferred unless a specific array of software for implementing such technical characteristics is clearly described otherwise. That is, the aforementioned various embodiments may be present, and may be partially modified while having the same technical features as those of the present disclosure, and thus such modified embodiments should also be regarded as falling within the scope of the present disclosure.

Furthermore, the flowchart describes operations in the drawing in a specific sequence, but has been shown to obtain the most preferred result, and it should not be understood that such operations must be carried out in the specific sequence or sequential sequence shown, or that all shown operations must be carried out. In a specific case, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

1. A sleep analysis segment detection method performed in a system including a layer unit, a detection unit, a memory unit, and a data analysis unit, the method comprising:
detecting a user's biosignal from the detection unit provided in the layer unit;
storing the user's biosignal detected by the detection unit in the memory unit; and
analyzing the user's biosignal stored in the memory unit by the data analysis unit to detect the user's sleep analysis segment.

2. The method of claim 1, wherein the detecting of the user's biosignal comprises:
performing detection on at least one information of a user's weight, height, body proportions, identification information, movement information, heart rate, and breathing state through the detection unit including at least one sensor among a pressure sensor, a vibration sensor, a piezoelectric sensor, an acceleration sensor, an acoustic sensor, a polyvinylidene film (PVDF) sensor, an electromechanical film (EMFi) sensor, a force sensing resistor (FSR) sensor, an infrared sensor, a motion sensor, and a facial recognition sensor.

3. The method of claim 1, wherein the detecting of the user's sleep analysis segment from the data analysis unit comprises:
a first sleep analysis segment detection step of detecting a time point when the user lies down on the layer unit and a time point when the user gets up and leaves the layer unit.

4. The method of claim 3, further comprising:
subsequent to the first sleep analysis segment detection step,
a second sleep analysis segment detection step of excluding the user's non-sleep segment from the first sleep analysis segment.

5. The method of claim 4, wherein the second sleep analysis segment detection step comprises:
determining a segment in which at least one of the user's heart rate, breathing state, and movement information detected by the detection unit exceeds a preset threshold value as a non-sleep segment, and excluding the non-sleep segment from the first sleep analysis segment.

6. The method of claim 4, further comprising:
subsequent to the second sleep analysis segment detection step,
a third sleep analysis segment detection step of excluding the user's activity segment from the second sleep analysis segment.

7. The method of claim 6, wherein the third sleep analysis segment detection step comprises:
determining a segment in which a signal value detected from the pressure sensor of the detection unit remains lower than a preset threshold value for above a preset time period as a user activity segment, and excluding the user activity segment from the second sleep analysis segment.

8. The method of claim 1, further comprising:
subsequent to the third sleep analysis segment detection step,
summing up, when there are at least two or more multiple independent sleep analysis segments within the third sleep analysis segment, the multiple sleep analysis segments.

9. A sleep analysis segment detection system, the system comprising:
at least one layer having a plurality of components;
a detection unit provided in the layer unit to detect a user's biosignal;
a memory unit in which the user's biosignal detected by the detection unit is stored; and
a data analysis unit that analyzes the user's biosignal stored in the memory unit to detect the user's sleep analysis segment.
